(19) United States
(12) Patent Application Publication
(10) Pub. No.: US 2013/0211090 A1
(43) Pub. Date: Aug. 15, 2013

(54) METHOD FOR THE PRODUCTION OF 5-FLUORO-1H-PYRAZOLO[3,4-B]PYRIDINE-3-CARBONITRILE

(71) Applicants: Markus Follmann, Koln (DE); Jens Ackerstaff, Berlin (DE)

(72) Inventors: Markus Follmann, Koln (DE); Jens Ackerstaff, Berlin (DE)

(73) Assignee: BAYER INTELLECTUAL PROPERTY GMBH, Monheim (DE)

(21) Appl. No.: 13/819,905

(22) PCT Filed: Aug. 31, 2011
(86) PCT No.: PCT/EP11/65004

§ 371 Date: Apr. 15, 2013

(30) Foreign Application Priority Data
Sep. 3, 2010 (DE) 102010040234.6

(51) Int. Cl. C07D 471/04

(52) U.S. Cl. CPC C07D 471/04
USPC 546/120; 546/119

(57) ABSTRACT

The present invention relates to a process for preparing 5-fluoro-1H-pyrazolo[3,4-b]pyridine-3-carbonitrile of the formula (I)

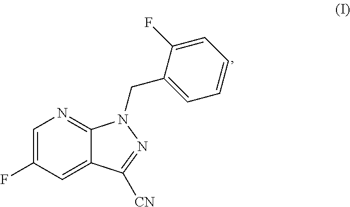 [SMILES: N#Cc1nn(Cc2ccccc2F)c2ncc(F)cc12]

which serves as a synthesis intermediate for production of medicaments, especially for production of medicaments for treatment and/or prophylaxis of cardiovascular disorders.

The present invention relates to a process for preparing 5-fluoro-1H-pyrazolo[3,4-b]pyridine-3-carbonitrile of the formula (I)

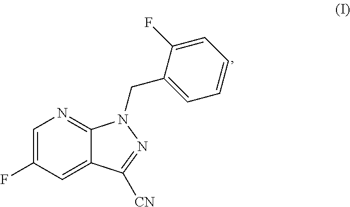 [SMILES: N#Cc1nn(Cc2ccccc2F)c2ncc(F)cc12]

which serves as a synthesis intermediate for production of medicaments, especially for production of medicaments for treatment and/or prophylaxis of cardiovascular disorders.

WO 2009/018415 describes the synthesis of 5-fluoro-1H-pyrazolo[3,4-b]pyridine-3-amine. Selective dechlorination of the nicotinic acid A to give the compound B, subsequent conversion to the amide C, the reduction thereof to the nitrile and the final cyclization with hydrazine hydrate form the 5-fluoro-1H-pyrazolo[3,4-b]pyridine core. Scheme 1 below illustrates the synthesis.

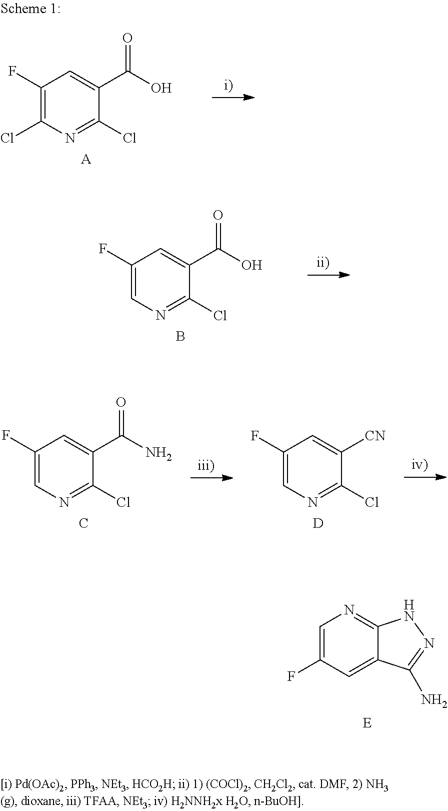

It is an object of the present invention to provide an efficient process with high yield for preparation of 5-fluoro-1H-pyrazolo[3,4-b]pyridine-3-carbonitrile of the formula (I)

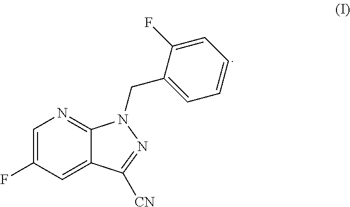 [SMILES: N#Cc1nn(Cc2ccccc2F)c2ncc(F)cc12]

This object is achieved in accordance with the present invention, as follows. Scheme 2 below illustrates the individual reaction steps.

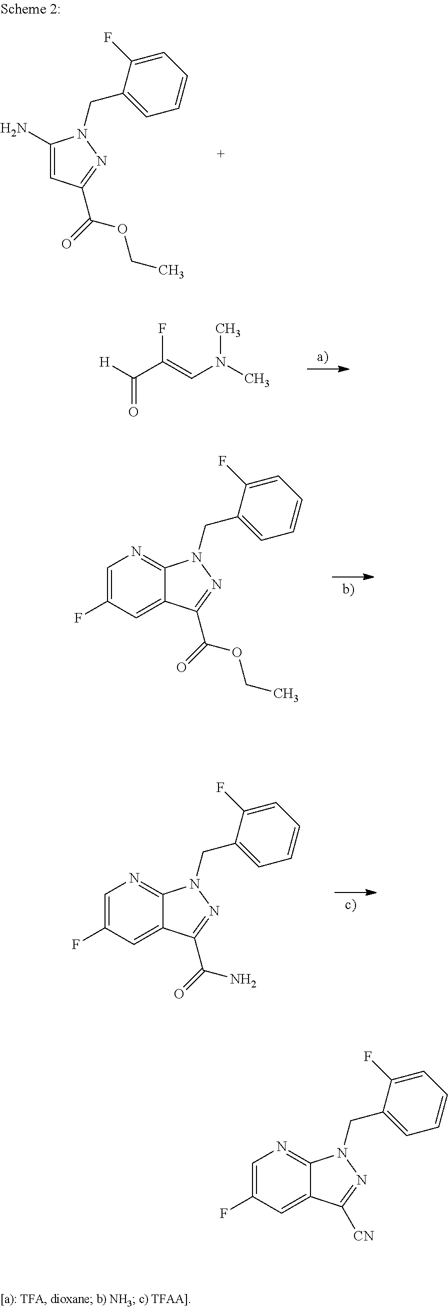

Specifically, the process according to the invention for preparing a compound of the formula (I)

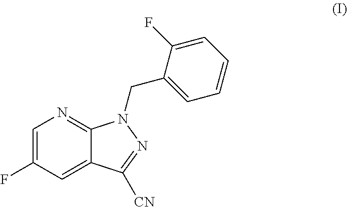 [SMILES: N#Cc1nn(Cc2ccccc2F)c2ncc(F)cc12]

comprises the cyclization of the 5-aminopyrazole derivative (II)

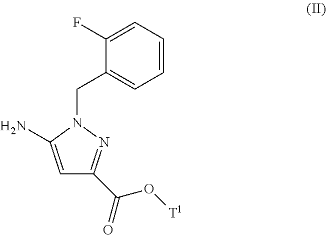

in which
T¹ is (C₁-C₄)-alkyl
in the presence of a suitable acid with the aldehyde (III)

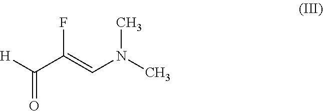

to give the ester of the formula (IV)

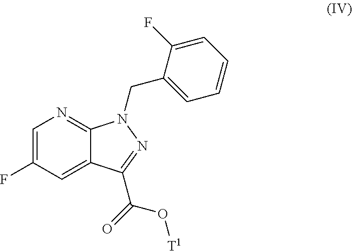

in which T¹ is as defined above,
the subsequent reaction thereof with ammonia to give the amide of the formula (V)

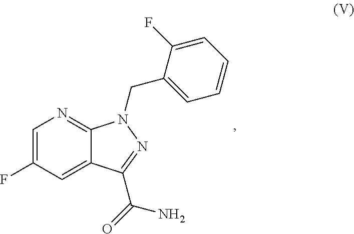 [SMILES: NC(=O)c1nn(Cc2ccccc2F)c2ncc(F)cc12]

and the subsequent dehydration to give the nitrile (I).

The compound of the formula (II) is known from the literature and can be prepared in analogy to example 20A in WO 00/06569.

The compound of the formula (III) is known from the literature and can be prepared as described in Justus Liebigs Ann. Chem. 1970, 99-107.

The cyclization of the 5-aminopyrazole derivative of the compound (II) with the aldehyde of the compound (III) to give the compound of the formula (IV) is effected in an inert solvent, optionally in the presence of trifluoroacetic acid, within a temperature range of +50° C. to +200° C., preferably at +80° C. to +140° C., at standard pressure, within, for example 10 to 80 hours, preferably within 48 to 72 hours.

Inert solvents are, for example, alcohols such as methanol, ethanol, n-propanol or iso-propanol, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions or other solvents, acetonitrile or N,N-dimethylformamide, or mixtures of solvents. Preference is given to dioxane.

The formation of the amide (IV)→(V) is effected by reaction in an inert solvent with ammonia within a temperature range of 0° C. to +50° C., preferably of +20° C. to +30° C., at standard pressure or elevated pressure, within 24 to 72 hours.

Inert solvents are, for example, alcohols such as methanol, ethanol, n-propanol or iso-propanol. Preference is given to using a solution of ammonia in methanol in a concentration of 5N to 7N.

The dehydration of the amide (V) to the nitrile (I) is effected in an inert solvent, in the presence of a suitable base, with a suitable dehydrating agent, for example trifluoroacetic anhydride, acetic anhydride or trifluoromethanesulfonic anhydride, within a temperature range of 0° C. to +60° C., preferably at +20° C. to +30° C., within 12 to 36 hours.

Preference is given to trifluoroacetic anhydride.

Inert solvents are ethers such as diethyl ether, dioxane, tetrahydrofuran (THF), glycol dimethyl ether or diethylene glycol dimethyl ether, hydrocarbons such as benzene, toluene, xylene, hexane, cyclohexane or mineral oil fractions or other solvents, acetonitrile or N,N-dimethylformamide, or mixtures of solvents. Preference is given to THF.

Suitable bases are, for example, organic amines such as triethylamine, diisopropylethylamine, pyridine, 1,8-diazabicyclo[5.4.0]undec-7-ene (DBU) or 1,5-diazabicyclo[4.3.0]non-5-ene (DBN). Preference is given to pyridine.

The compounds described in the context of the process according to the invention may also be in the form of the salts, solvates or solvates of the salts thereof.

The compounds described in the context of the process according to the invention may, depending on the structure, also be in the form of the tautomers thereof.

Preferred salts in the context of the invention are physiologically acceptable salts of the compounds used and prepared in the process according to the invention.

Physiologically acceptable salts of the compounds used and prepared in the process according to the invention include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, malic acid, citric acid, fumaric acid, maleic acid and benzoic acid.

Physiologically acceptable salts of the compounds used and prepared in the process according to the invention also include salts of customary bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dihydroabietylamine, arginine, lysine, ethylenediamine and methylpiperidine.

In the context of the invention, solvates refer to those forms of the compounds used and prepared in the process according to the invention which, in the solid or liquid state, form a complex by coordination with solvent molecules. Hydrates are a specific form of the solvates in which the coordination is with water.

In the context of the present invention, the substituents, unless specified otherwise, are each defined as follows:

Alkyl in the context of the invention is a linear or branched alkyl radical having 1 to 4 carbon atoms. Preferred examples include: methyl, ethyl, n-propyl, isopropyl, n-butyl, iso-butyl, sec-butyl and tert-butyl.

The present invention is illustrated in detail below by non-limiting preferred examples and comparative examples. Unless stated otherwise, all amounts given refer to percentages by weight.

### A. EXAMPLES

### Abbreviations:

Ac acetyl
CI chemical ionization (in MS)
DCI direct chemical ionization (in MS)
DMF dimethylformamide
DMSO dimethyl sulfoxide
eq. equivalent(s)
ESI electrospray ionization (in MS)
Et ethyl
GC/MS gas chromatography-coupled mass spectrometry
sat. saturated
h hour(s)
HPLC high-pressure high-performance liquid chromatography
HV high vacuum
conc. concentrated
LC/MS liquid chromatography-coupled mass spectrometry
Me methyl
min minute(s)
MS mass spectrometry
NMR nuclear magnetic resonance spectrometry
rac racemic/racemate
R_{f} retention factor (in thin layer chromatography on silica gel)
RT room temperature
Rₜ retention time (in HPLC)
SFC supercritical fluid chromatography
THF tetrahydrofuran
UV ultraviolet spectrometry
v/v volume to volume ratio (of a solution)

### LC/MS, HPLC and GC/MS Methods:

### Method 1 (LC-MS):

Instrument: Waters ACQUITY SQD UPLC System; column: Waters Acquity UPLC HSS T3 1.8μ 50×1 mm; eluent A: 1 1 water+0.25 ml 99% formic acid, eluent B: 1 1 acetonitrile+0.25 ml 99% formic acid; gradient: 0.0 min 90% A→1.2 min 5% A→2.0 min 5% A; oven: 50° C.; flow rate: 0.40 ml/min; UV detection: 210-400 nm.

### WORKING EXAMPLES

### Example 1

### Ethyl 5-fluoro-1-(2-fluorobenzyl)-1H-pyrazolo[3,4-b]pyridine-3-carboxylate

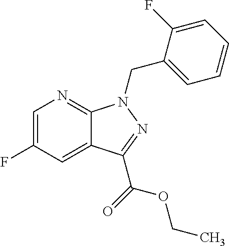 [SMILES: CCOC(=O)c1nn(Cc2ccccc2F)c2ncc(F)cc12]

13.487 g (51.228 mmol) of ethyl 5-amino-1-(2-fluorobenzyl)-1H-pyrazole-3-carboxylate (preparation described for example 20A in WO 00/06569) were initially charged in 300 ml of dioxane, and 6 g (51.228 mmol) of 3-(dimethylamino)-2-fluoroacrylaldehyde (preparation described in Justus Liebigs Annalen der Chemie 1970; 99-107) were added at RT. Subsequently, 4.736 ml (61.473 mmol) of trifluoroacetic acid were added and the mixture was heated to reflux while stirring for 3 days. After cooling, the mixture was concentrated under reduced pressure, and water and ethyl acetate were added to the residue. The phases were separated and the organic phase was washed twice with water. The combined aqueous phases were subsequently extracted twice with ethyl acetate. The combined organic phases were dried over sodium sulfate, filtered and concentrated under reduced pressure. The residue (22 g) was subsequently purified by chromatography on silica gel (eluent: dichloromethane). This gave 5.67 g (35% of theory) of the title compound.

LC-MS (method 1): Rₜ=1.17 min

MS (ESIpos): m/z=318 (M+H)⁺

¹H NMR (400 MHz, DMSO-d₆): δ=1.37 (t, 3H), 4.40 (q, 2H), 5.86 (s, 2H), 7.15-7.27 (m, 3H), 7.36-7.41 (m, 1H), 8.25 (d, 1H), 8.78 (s br., 1H).

### Example 2

### 5-Fluoro-1-(2-fluorobenzyl)-1H-pyrazolo[3,4-b]pyridine-3-carboxamide

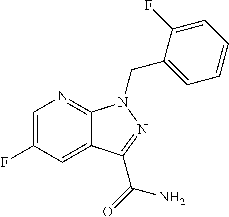 [SMILES: NC(=O)c1nn(Cc2ccccc2F)c2ncc(F)cc12]

1.00 g (3.152 mmol) of the compound obtained in example 1 was stirred in 10 ml of a 7N solution of ammonia in methanol at RT for three days. This was followed by concentration under reduced pressure. This gave 908 mg (99% of theory) of the title compound.

LC-MS (method 1): Rₜ=0.85 min

MS (ESIpos): m/z=289 (M+H)⁺

¹H NMR (400 MHz, DMSO-d₆): δ=5.87 (s, 2H), 7.12-7.26 (m, 3H), 7.34-7.40 (m, 1H), 7.60 (s br., 1H), 7.87 (s br., 1H), 8.28 (dd, 1H), 8.72 (dd, 1H).

### Example 3

### 5-Fluoro-1-(2-fluorobenzyl)-1H-pyrazolo[3,4-b]pyridine-3-carbonitrile

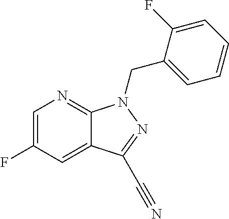 [SMILES: N#Cc1nn(Cc2ccccc2F)c2ncc(F)cc12]

900 mg (3.122 mmol) of the compound obtained in example 2 were dissolved in THF (14 ml), and 0.646 ml (7.993 mmol) of pyridine was added. Thereafter, 1.129 ml (7.993 mmol) of trifluoroacetic anhydride were slowly added dropwise and then the mixture was stirred at RT overnight. Thereafter, the reaction mixture was poured onto water and extracted three times with ethyl acetate. The combined organic phases were extracted with saturated aqueous sodium hydrogencarbonate solution and 1N hydrochloric acid, and then washed with saturated aqueous sodium chloride solution. The organic phase was dried over sodium sulfate, filtered and concentrated. This gave 850 mg (99% of theory) of the title compound.

LC-MS (method 1): Rₜ=1.06 min

MS (ESIpos): m/z=271 (M+H)⁺

¹H NMR (400 MHz, DMSO-d₆): δ=5.87 (s, 2H), 7.17-7.42 (m, 4H), 8.52 (dd, 1H), 8.87 (dd, 1H).

1. A process for preparing a compound of the formula (I) 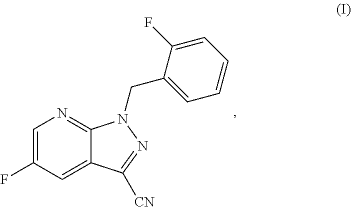 [SMILES: N#Cc1nn(Cc2ccccc2F)c2ncc(F)cc12]
comprising dehydrating the amide of the formula (V)

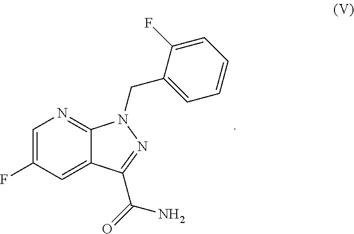 [SMILES: NC(=O)c1nn(Cc2ccccc2F)c2ncc(F)cc12]

2. The process of claim 1, further comprising preparing the compound of formula (V) by reacting an ester of the formula (IV) 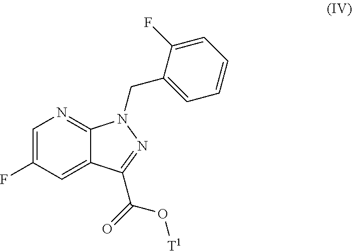
in which
T¹ is (C₁-C₄)-alkyl
with ammonia.

3. The process of claim 2, further comprising preparing the ester of the formula (IV) by cyclizing the 5-aminopyrazole derivative (II) 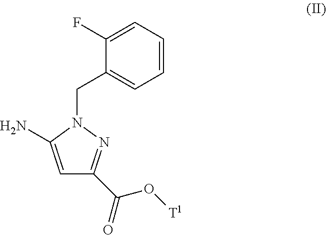
in which
T¹ is (C₁-C₄)-alkyl
in the presence of a suitable acid with the aldehyde (III)

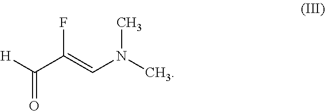

4. A compound of the formula (V) 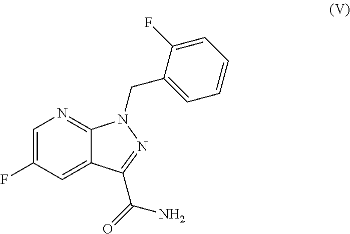 [SMILES: NC(=O)c1nn(Cc2ccccc2F)c2ncc(F)cc12]
and the salts, solvates and solvates of the salts thereof.

5. A compound of the formula (IV) 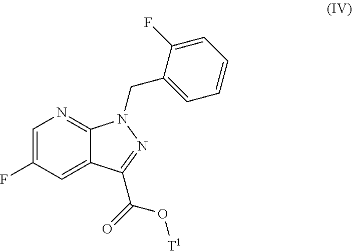
in which T¹ is as defined above
and the salts, solvates and solvates of the salts thereof.
